(19) United States
(12) Patent Application Publication
(10) Pub. No.: US 2026/0198874 A1
(43) Pub. Date: Jul. 16, 2026

(54) SCAN SUBJECT SUPPORT APPARATUS, DOCKING APPARATUS, AND MEDICAL IMAGING SYSTEM

(71) Applicant: GE Precision Healthcare LLC, Waukesha, WI (US)

(72) Inventors: Yigang Xia, Beijing (CN); Qian Xu, Beijing (CN); Yifei Dong, Beijing (CN)

(21) Appl. No.: 19/449,066

(22) Filed: Jan. 14, 2026

(30) Foreign Application Priority Data
Jan. 14, 2025 (CN) 202520081791.9

(51) Int. Cl. A61B 6/04; A61B 6/00

(52) U.S. Cl. CPC A61B 6/0487; A61B 6/0407; A61B 6/4435

(57) ABSTRACT

Embodiments of the present application provide a scan subject support apparatus, a docking apparatus, and a medical imaging system. The scan subject support apparatus includes a support platform for a subject, a lifting drive portion for the support platform, and a base for the lifting drive portion. The scan subject support apparatus further includes a moving portion, mounted on the base and capable of moving the entire scan subject support apparatus, and a connecting portion, provided on the base and including a connecting hole or a connecting rod provided in a longitudinal direction of the scan subject support apparatus. The connecting hole or the connecting rod is connected to a docking apparatus on a side of a scanning apparatus. Therefore, the scan subject support apparatus and the scanning apparatus can be connected, and the subject can be balanced, thereby improving the stability of the scan subject support apparatus.

### CROSS-REFERENCE TO RELATED APPLICATIONS

This application claims priority to Chinese Application No. 202520081791.9, filed on January 14, 2025, the disclosure of which is incorporated herein by reference in its entirety.

### TECHNICAL FIELD

The embodiments of the present application relate to the technical field of medical devices, and in particular, to a scan subject support apparatus, a docking apparatus, and a medical imaging system.

### BACKGROUND

A scanning apparatus is an important component in a medical imaging system, which scans a patient in a non-invasive manner to acquire a medical image of an anatomical tissue of interest of the patient, thereby assisting a doctor with diagnosis. For example, computed tomography (CT) utilizes accurately collimated X-ray beams, gamma rays, etc., together with a highly sensitive detector, to perform cross-sectional scans one by one around a certain site of a human body, which is characterized by a short scanning time and image clarity, and can be used to detect a variety of diseases.

A scan subject support apparatus is another important component in the medical imaging scanning system, and is required to lift and lower a scan subject and transport the scan subject to a scanning position in the scanning apparatus. The scan subject support apparatus includes a support platform. During scanning, the support platform may move toward the scanning apparatus (a movement direction may be referred to as a moving-out direction) to move the scan subject to a suitable scanning position. After scanning is completed, the support platform may move in a direction opposite to the moving-out direction to bring the scan subject back to an original position.

Usually, the scan subject (for example, a patient) needs to lie on the support platform or leave the support platform during scanning. However, an additional measure (for example, a

stretcher) is required to assist a scan subject with limited mobility (for example, a critically ill or paralyzed patient, or a patient that has a fracture) in getting on and off the support platform. Such a measure may cause harm to the scan subject. In another measure, a movable scan subject support apparatus or a movable scanning apparatus may be used to move the two together during imaging scanning.

### SUMMARY

The inventors have found that during scanning, a support platform of a movable scan subject support apparatus bears a scan subject and moves and extends into a scanning apparatus. The weight of the support platform and the scan subject deviates from a main body of the scan subject support apparatus, that is, the center of gravity of the entire scan subject support apparatus changes. In this case, it is difficult to balance the load of the scan subject, that is, a side of the scan subject support apparatus close to the scanning apparatus may tilt. Consequently, the stability of the scan subject support apparatus cannot be effectively maintained, and the imaging quality of imaging scanning is further affected. On the other hand, it may be difficult for an operator to move the movable scan subject support apparatus to a suitable position, that is, the extending support platform may collide with the scanning apparatus or may be too close to a certain side of the scanning apparatus, requiring the scan subject support apparatus to be re-positioned or aligned with the scanning apparatus, which affects the efficiency or imaging quality of an imaging scanning operation.

In response to at least one of the above technical problems or other similar problems, the embodiments of the present application provide a scan subject support apparatus, a docking apparatus, and a medical imaging system. Even if a movable scan subject support apparatus is used, correct docking with the scanning apparatus can be implemented, so that the load of a scan subject can be balanced, thereby effectively maintaining the stability of the scan subject support apparatus.

According to a first aspect of the embodiments of the present application, provided is a scan subject support apparatus, including a support platform for bearing a scan subject, a lifting drive portion for bearing the support platform, and a base for bearing the lifting drive portion.

The scan subject support apparatus includes a moving portion, mounted on the base and capable of moving the entire scan subject support apparatus; and a connecting portion, provided on the base, the connecting portion incl a connecting hole or a connecting rod provided in a longitudinal direction of the scan subject support apparatus; the connecting hole or the connecting rod being capable of being fixedly connected to a docking apparatus fixed to a side of a scanning apparatus.

In some embodiments, when the scan subject support apparatus moves toward the scanning apparatus, a connecting rod of the docking apparatus is inserted into the connecting hole of the scan subject support apparatus; and when the scan subject support apparatus is separated from the scanning apparatus, the connecting rod of the docking apparatus leaves the connecting hole of the scan subject support apparatus;

or, when the scan subject support apparatus moves toward the scanning apparatus, the connecting rod of the scan subject support apparatus is inserted into a connecting hole of the docking apparatus; and when the scan subject support apparatus is separated from the scanning apparatus, the connecting rod of the scan subject support apparatus leaves the connecting hole of the docking apparatus.

In some embodiments, at least two connecting holes or connecting rods are provided in a transverse direction of the scan subject support apparatus, and the connecting holes or the connecting rods have a length that exceeds a predetermined threshold, so as to fix the scan subject support apparatus in the transverse direction and a vertical direction of the scan subject support apparatus.

In some embodiments, the moving portion includes a plurality of casters disposed on a lower side of the base; the plurality of casters include a first caster located on a side of the scan subject support apparatus close to the scanning apparatus during scanning and a second caster located on a side away from the scanning apparatus; and the connecting portion is disposed at an end of the base close to the scanning apparatus.

In some embodiments, the scan subject support apparatus further includes a fixing portion, located at an end of the base away from the scanning apparatus. The fixing portion including a fixing rod disposed in a vertical direction and a pressing apparatus capable of moving the fixing rod in the vertical direction.

In some embodiments, the pressing apparatus includes a pedal, moving the fixing rod by means of a rotating shaft, and a guide piece, guiding movement of the fixing rod.

In some embodiments, the ground is provided with a fixing hole; when the pressing apparatus is pressed, the fixing rod is inserted into the fixing hole to fix the scan subject support apparatus in the longitudinal direction; and when the pressing apparatus is reset to a non-pressed state, the fixing rod is separated from the fixing hole.

According to a second aspect of the embodiments of the present application, provided is a docking apparatus, connecting a scan subject support apparatus and a scanning apparatus; the docking apparatus including a main body portion, fixed to the ground or the scanning apparatus, and having a connecting rod or a connecting hole provided in a longitudinal direction of the scan subject support apparatus, the connecting hole or the connecting rod being capable of being fixedly connected to a connecting hole or a connecting rod of the scan subject support apparatus.

In some embodiments, the connecting rod includes a cylindrical main body portion and a conical head portion; and at least two connecting holes or connecting rods are provided in a transverse direction of the docking apparatus.

In some embodiments, the docking apparatus further includes a camera, located at a front end of the connecting rod or in the connecting hole, wherein an image acquired by the camera is displayed on a display portion, so as to guide movement of the scan subject support apparatus.

In some embodiments, the docking apparatus further includes a docking switch, located on the main body portion, wherein when the scan subject support apparatus and the scanning apparatus are successfully docked, the docking switch is triggered.

According to a third aspect of the embodiments of the present application, provided is a medical imaging system. The system includes a scanning device, the foregoing scan subject support apparatus, and the foregoing docking apparatus.

Beneficial effects of the embodiments of the present application are as follows: The load of a scan subject after at least a part of which is moved out from the support platform can be balanced, thereby improving the stability of the support apparatus, and further improving the imaging quality of imaging scanning. Further, the scan subject support apparatus can be accurately moved to a suitable position, preventing the extending support platform from colliding with the scanning apparatus or being too close to a certain side of the scanning apparatus, which thus affects the efficiency or imaging quality of an imaging scanning operation, and also preventing thus required repositioning of the scan subject support apparatus or alignment with the scanning apparatus.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application include many changes, modifications, and equivalents.

The features described and/or illustrated for one implementation may be used in one or more other implementations in the same or similar way, be combined with features in other implementations, or replace features in other implementations.

It should be emphasized that the terms “include/comprise” when used herein refer to the presence of features, integrated components, steps, or assemblies, but do not preclude the presence or addition of one or more other features, integrated components, steps, or assemblies.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT device according to an embodiment of the present application;

FIG. 2 is a schematic diagram of a CT imaging system according to an embodiment of the present application;

FIG. 3 is a schematic diagram of the composition of a medical imaging system according to an embodiment of the present application;

FIG. 4 is a schematic diagram of a scan subject support apparatus according to an embodiment of the present application;

FIG. 5 is a schematic diagram of a movable support portion in which a platform is moved out according to an embodiment of the present application;

FIG. 6 is a schematic diagram of a local part of a scan subject support apparatus according to an embodiment of the present application;

FIG. 7 is a schematic diagram of a connecting portion of a scan subject support apparatus according to an embodiment of the present application;

FIG. 8 is a schematic diagram of a docking apparatus according to an embodiment of the present application; and

FIG. 9 is a schematic diagram of docking of a medical imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to...” and the term “based on” should be construed as “at least in part based on...”, unless otherwise specified in the context.

The features described and/or illustrated for one implementation may be used in one or more other implementations in the same or similar way, be combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

The scanning apparatus (that is, a medical imaging scanning system or an imaging scanning apparatus) described in the present application may be applicable to a variety of medical imaging modalities, including, but not limited to: computed tomography (CT), magnetic resonance imaging (MRI), C-arm imaging, positron emission computed tomography (PET), single photon emission computed tomography (SPECT), or any other suitable medical imaging device.

For example, the embodiments of the present application are described below in conjunction with an X-ray computed tomography (CT) imaging system. Those skilled in the art will understand that the embodiments of the present application can also be applicable to other medical imaging scanning systems.

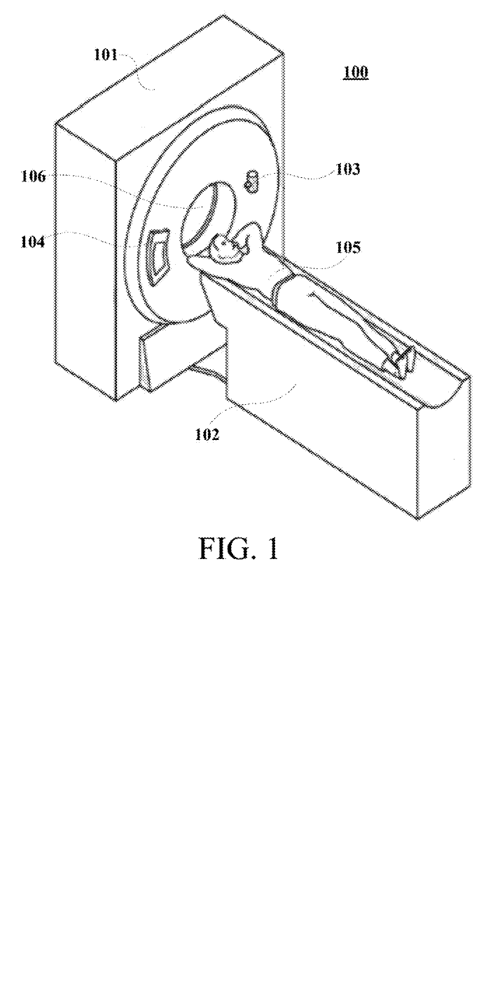

FIG. 1 is a schematic diagram of a CT device according to an embodiment of the present application, and schematically shows a CT device 100. As shown in FIG. 1, the CT device 100 includes a scanning gantry 101 and a patient table 102 (for example, a scanning

table). The scanning gantry 101 has an X-ray source 103, and the X-ray source 103 projects an X-ray beam toward a collimator or detector assembly 104 on the opposite side of the scanning gantry 101. A subject under examination (also referred to as a scan subject) 105 may lie on the patient table 102 and be moved into a scanning gantry opening 106 along with the patient table 102. Medical image data of the subject under examination 105 can be obtained through scanning performed by the X-ray source 103.

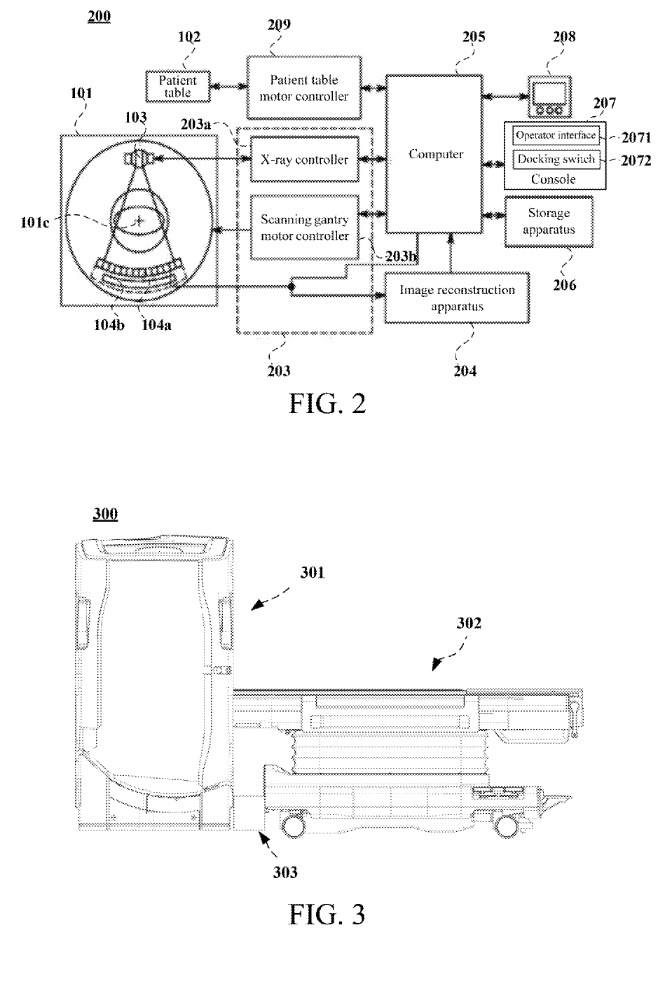

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the subject under examination 105.

The DAS 104b, based on the sensing of the detector units 104a, converts collected information into projection data for subsequent processing. During the scanning for acquiring the X-ray projection data, the scanning gantry 101 and components mounted thereon rotate around a center of rotation 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and executes image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface 2071 in a certain form, such as a keyboard, a mouse, or a speech activated controller. The console 207 may further have an input apparatus such as a docking switch 2072. In addition, the console 207 may have another suitable input apparatus. An associated display 208 allows the operator to observe a reconstructed image and other data from the computer 205. The commands and parameters provided by the operator are used by the computer 205 to provide control signals and

information to the DAS 104b, the X-ray controller 203a, and the scanning gantry motor controller 203b. In addition, the computer 205 operates a patient table motor controller 209, which controls the patient table 102 to position the subject under examination 105 and the scanning gantry 101. In particular, the patient table 102 moves the subject under examination 105 to completely or partially pass through the scanning gantry opening 106 in FIG. 1.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. The storage device may be located in the medical imaging device, in a server outside the medical imaging device, in an independent medical imaging storage system (such as a picture archiving and communication system (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical imaging scanning is schematically described above, and the embodiments of the present application are described in detail below with reference to the drawings. In the embodiments described below, a medical imaging scanning device being a CT device is used as an example for description, and the content of the description is also applicable to other medical devices.

FIG. 3 is a schematic diagram of the composition of a medical imaging system according to an embodiment of the present application. As shown in FIG. 3, a medical imaging system 300 includes a scanning apparatus 301, a movable scan subject support apparatus 302, and a docking apparatus 303. The scan subject support apparatus 302 is connected to the scanning apparatus 301 by means of the docking apparatus 303, to connect the scan subject support apparatus 302 and the scanning apparatus 301 together.

In some embodiments, the medical imaging system 300 may further include other components not shown. For details, refer to FIG. 1 and FIG. 2. In addition, for the scanning apparatus 301, also refer to FIG. 1 and FIG. 2 or refer to the related art.

The following further describes the medical imaging system 300 from the side of the scan subject support apparatus 302.

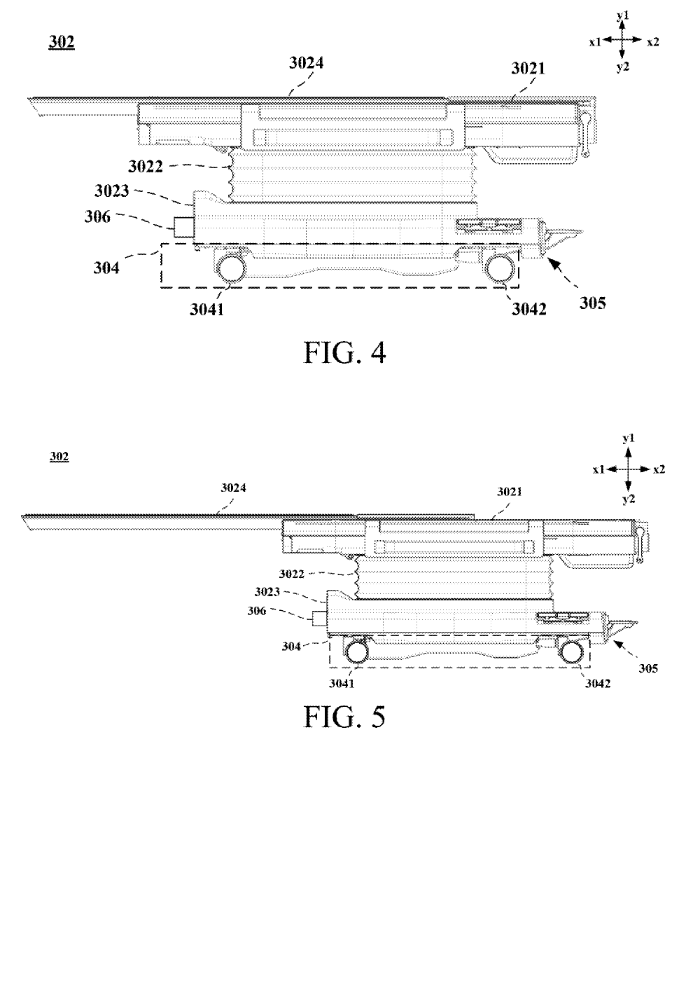

FIG. 4 is a schematic diagram of a scan subject support apparatus according to an embodiment of the present application. As shown in FIG. 4, the scan subject support apparatus 302 includes a support platform 3021 for bearing a scan subject, a lifting drive portion 3022 for bearing the support platform 3021, a base 3023 for bearing the lifting drive portion 3022, a moving portion 304 mounted on the base 3023 and capable of moving the entire scan subject support apparatus 302, a fixing portion 305 (only some components are shown in FIG. 4) for fixing the scan subject support apparatus 302, and a connecting portion 306 (only the position thereof is schematically shown in FIG. 4) for docking with the docking apparatus 303.

The support platform 3021 is configured to bear the scan subject, the scan subject may lie on an upper surface of the support platform 3021, and the position of the scan subject relative to the scanning apparatus 301 is adjusted by moving the support platform 3021. The support platform 3021 has a movable support portion 3024, which can move in a length direction of the support platform 3021, that is, move in an x-axis direction (the x1 direction and the x2 direction shown in FIG. 4), to adjust the position of the scan subject in the x-axis direction, so that the scan subject is placed at a scanning position of the scanning apparatus 301. As shown in FIG. 4, the movable support portion 3024 is at an initial position. In this case, the scan subject lying on the movable support portion 3024 is within a range of the support platform 3021 in the up-down direction.

FIG. 5 is another schematic diagram of a scan subject support apparatus according to an embodiment of the present application. As shown in FIG. 5, the movable support portion 3024 may move in the x1 direction, and at least a part thereof moves beyond the range of the support platform 3021 in the up-down direction, to move the scan subject to the scanning position. After scanning ends, the movable support portion 3024 may move in the x2 direction to return to the initial position.

In the embodiments of the present application, the scan subject support apparatus 302 may also be referred to as a “scanning table”, a “medical bed”, a “patient table”, or the like, and the movable support portion 3024 of the support platform 3021 may also be referred to as a “bed plate”. This is not limited in the present application. The support platform 3021 is in the shape of a rectangular plate with a uniform thickness, and is configured to bear the scan subject, for example, the scan subject lies on the support platform 3021 in a supine posture.

In the embodiments of the present application, the movable support portion 3024 may also move the scan subject in a longitudinal direction (that is, the x-axis direction) of the support platform 3021. The support platform 3021 may be made of a material having low X-ray attenuation, for example, made of a carbon fiber composite material. The embodiments of the present application are not limited thereto.

As shown in FIG. 4 and FIG. 5, the lifting drive portion 3022 is configured to bear the support platform 3021, and can adjust the vertical height of the support platform 3021 to adjust the position of the scan subject in a vertical direction, that is, the position in a y-axis direction (the y1 direction and the y2 direction shown in FIG. 4).

As shown in FIG. 4 and FIG. 5, the moving portion 304 is mounted on the base 3023 and can move the entire scan subject support apparatus 302. The moving portion 304 includes a plurality of casters disposed on a lower side of the base 3023. The plurality of casters include a first caster 3041 located on a side (that is, a side of the scan subject support apparatus 302 close to the scanning apparatus 301 during scanning) of a moving-out direction (the x1 direction shown in FIG. 4) in which the support platform 3021 moves toward the scanning apparatus 301 and a second caster 3042 located on a side (that is, a side of the scan subject support apparatus

302 away from the scanning apparatus 301 during scanning) opposite to a movement direction. That is, the first caster 3041 is located on the side of the x1 direction of the base 3023 or the side close to the scanning apparatus 301, and the second caster 3042 is located on the side of the x2 direction of the base 3023 or the side away from the scanning apparatus 301.

In this embodiment of the present application, as shown in FIG. 5, after at least a part of the scan subject is moved out from the support platform, the center of gravity of the entire movable scan subject support apparatus 302 changes. The center of gravity may deviate from the center of the scan subject support apparatus 302 and lean toward the first caster 3041. In this case, it is difficult to balance the load of the scan subject, causing the scan subject support apparatus 302 to tilt. In addition, it may be difficult for an operator to move the scan subject support apparatus 302 to a suitable position, that is, the extending support platform 3021 may collide with the scanning apparatus 301 or may be too close to a certain side of the scanning apparatus 301, thus requiring repositioning or otherwise affecting the quality of an image acquired in subsequent imaging scanning.

In this embodiment of the present application, the scan subject support apparatus 302 and the scanning apparatus 301 can be effectively connected by means of the connecting portion 306, and the stability of the scan subject support apparatus can be maintained, so that the occurrence of docking failure is avoided, thereby improving the efficiency and imaging quality of an imaging scanning operation. Further, in this embodiment of the present application, the scan subject support apparatus 302 may be further fixed by means of the fixing portion 305, to further improve the stability of the scan subject support apparatus 302.

The following further exemplarily describes the fixing portion 305. The fixing portion 305 in the present disclosure is optional. For example, only the connecting portion 306 is provided and no fixing portion 305 is provided, or other means such as electromagnetic attraction, bonding, snapping, etc., may be used for the fixing portion 305. The present application is not limited thereto.

In some embodiments, the fixing portion 305 is located at one end of the base 3023 away from the scanning apparatus 301, and includes a fixing rod 3051 disposed on the base 3023 and disposed in a vertical direction (that is, perpendicular to the base 3023 or perpendicular to the ground), and a fixing hole 3052 provided at a position to be fixed of the scan subject support apparatus 302. By means of inserting the fixing rod 3051 into the fixing hole 3052, the scan subject support apparatus 302 can be fixed at the position to be fixed, and the scan subject support apparatus 302 can be prevented from moving along the x-axis. The following uses an example in which the position to be fixed is located on the ground for description. The present application is not limited thereto.

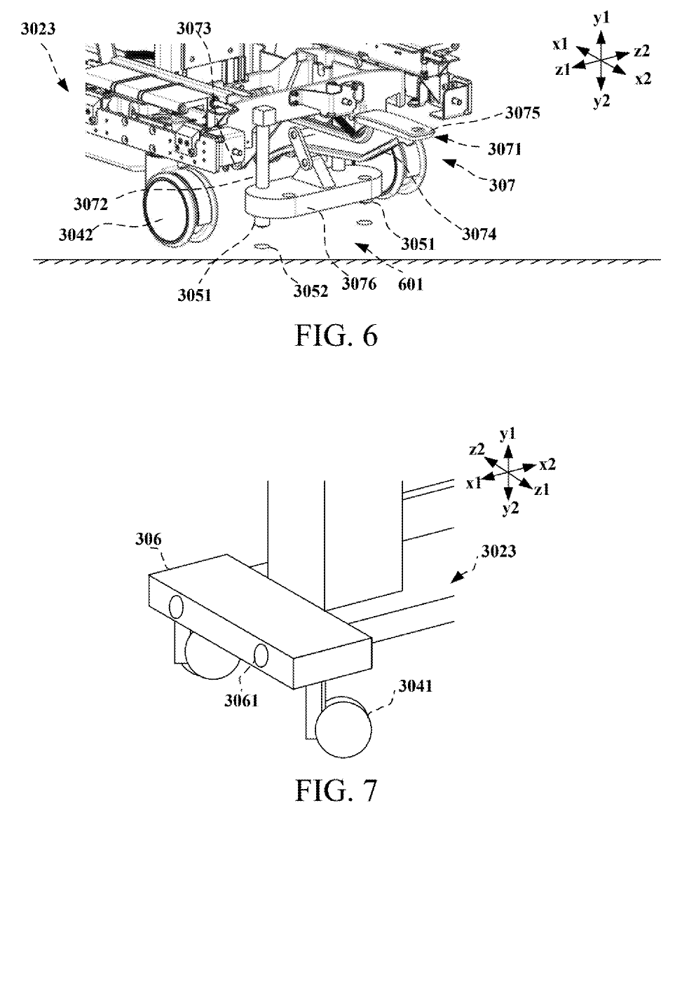

FIG. 6 is a schematic diagram of a local part of a scan subject support apparatus according to an embodiment of the present application, and exemplarily shows some components of the fixing portion 305. As shown in FIG. 6, a fixing rod 3051 is disposed on a side of the base 3023 that faces the position to be fixed 601 (that is, the lower side of the base 3023 that faces the ground), and is located at an end of the second caster 3042 (that is, an end away from the scanning apparatus 301), that is, the fixing rod 3051 is disposed on the side of the y2 direction of the base 3023 (the lower side), and located on a side of the x2 direction of the second caster 3042 (the right side). The fixing hole 3052 is provided on the ground and located at the position to be fixed 601 of the scan subject support apparatus 302.

For example, if the position of the fixing rod 3051 corresponds to the position of the fixing hole 3052 in an up-down direction (that is, the y1-y2 direction in FIG. 6), the fixing rod 3051 is inserted into the fixing hole 3052. In this case, the scan subject support apparatus 302 is fixed at the position to be fixed 601 by means of the action of a moment of force, and is prevented from moving in the x-axis direction and the z-axis direction.

In another example, if the positions of the fixing rod 3051 and the fixing hole 3052 in the up-down direction do not correspond to each other, the fixing rod 3051 cannot be inserted into the fixing hole 3052, and in this case, the position of the scan subject support apparatus 302 needs to be adjusted, so as to ensure that the fixing rod 3051 can be inserted into the fixing hole 3052.

Therefore, after docking between the scan subject support apparatus 302 and the docking apparatus 303 is completed, the scan subject support apparatus 302 can be further fixed by means of the fixing rod 3051 and the fixing hole 3052, preventing scanning from starting before the scan subject support apparatus 302 fixed, thereby further improving safety.

In some embodiments, the fixing rod 3051 may be made of metal, such as steel, iron, etc., or other materials. The present application is not limited thereto.

In some embodiments, the number of fixing rods 3051 is not less than two, and the number of fixing holes 3052 is not less than the number of fixing rods 3051. For example, two fixing rods 3051 are disposed in a transverse direction of the scan subject support apparatus 302, that is, two fixing rods 3051 are disposed in the z-axis direction of the scan subject support apparatus 302, two fixing holes 3052 are also provided on the ground in the z-axis direction, and the fixing rods 3051 can be inserted into the fixing holes 3052. The present application is not limited thereto. The plurality of fixing rods 3051 and fixing holes 3052 may also be arranged in the longitudinal direction, that is, the x-axis direction, or in other directions. The present application is not limited thereto.

In some embodiments, the fixing portion 305 further includes a pressing apparatus 307 that moves the fixing rod 3051 in the vertical direction (that is, a direction perpendicular to the ground). The pressing apparatus 307 is located on a lower side of the scan subject support apparatus 302, that is, on the side of the y2 direction. Therefore, the fixing rod 3051 can be inserted into the fixing hole 3052 by means of the pressing apparatus 307, further improving the convenience of operation.

As shown in FIG. 6, for example, the pressing apparatus 307 includes a pedal 3071 and a guide piece 3072. The pedal 3071 is mounted on the base 3023 and located on the side of the x2 direction of the base 3023. The pedal 3071 is of a three stage structure, and includes a rotating shaft connecting portion 3073 rotatably connected to the base 3023, a pedal portion 3075 for an operator to press or step on, and a middle portion 3074 connecting the rotating shaft connecting portion 3073 and the pedal portion 3075. The middle portion 3074 is connected to an end surface of the base 3023 by means of a spring and is configured to reset the pedal 3071. The middle portion 3074 of the pedal 3071 is connected to a fixing portion 3076 carrying the fixing rod 3051 by means of a plate-shaped connecting rod, and a double-action release structure (not shown) may be disposed for the two, that is, the first time a user steps down on the pedal, the fixing portion 3076 of the fixing rod 3051 moves downward and the fixing rod 3051 is inserted into the fixing hole 3052 to be in a fixed position, and the second time the user steps down on the pedal 3071, the pedal 3071 moves out of the fixing hole 3052 under the action of the spring to return to a released position. When the pedal 3071 is pressed by an operator, the fixing rod 3051 may be moved toward the fixing hole 3052 by means of a rotating shaft. The guide piece 3072 guides the fixing rod 3051, limits a direction in which the fixing rod 3051 moves toward the fixing hole 3052, or limits a direction in which the fixing rod 3051 moves away from the fixing hole 3052.

In some embodiments, when the pressing apparatus 307 is pressed, the fixing rod 3051 is inserted into the fixing hole 3052 to fix the scan subject support apparatus 302 in the longitudinal direction (x-axis direction) and the transverse direction (z-axis direction). When the pressing apparatus 307 is restored to a non-pressed state, the fixing rod 3051 is separated from the fixing hole 3052, to release limits on the scan subject support apparatus 302 in the longitudinal direction (x-axis direction) and the transverse direction (z-axis direction).

For example, the guide piece 3072 is two posts shown in FIG. 6. The guide piece 3072 limits the fixing rod 3051 to move toward the fixing hole 3052 in the y2 direction (downward movement), or limits the fixing rod 3051 to move away from a second anchoring portion 1052 in the y1 direction (upward movement). The embodiments of the present application are not limited thereto.

Therefore, by means of the pedal 3071 and the guide piece 3072, the scan subject support apparatus can be fixed by using a simple structure, and the fixing rod 3051 can be inserted into the fixing hole 3052. In addition, by means of a restoring piece (for example, a spring), the fixing rod 3051 can be separated from the fixing hole 3052, to restore the pressing apparatus 307 to the non-pressed state, thereby further improving the convenience of operation.

The above exemplarily describes the fixing portion 305 of the present application, and the following further exemplarily describes the connecting portion 306.

FIG. 7 is a schematic diagram of a connecting portion of a scan subject support apparatus according to an embodiment of the present application. As shown in FIG. 7, the connecting portion 306 is disposed at an end of the base 3023 close to the scanning apparatus 301 and is located above the first caster 3041, that is, the connecting portion 306 is disposed on the side of the x1 direction of the base 3023 and is located on the side of the y1 direction of the first caster 3041. The connecting portion 306 includes a connecting hole or a connecting rod disposed in the longitudinal direction of the scan subject support apparatus 302, and the connecting hole or the connecting rod can be fixedly connected to the docking apparatus fixed to the scanning apparatus. FIG. 7 only schematically shows the connecting hole 3061. The present application is not limited thereto.

In some embodiments, at least two connecting holes or connecting rods are provided in the transverse direction (that is, the z-axis direction) of the scan subject support apparatus 302, or at least two connecting holes or connecting rods are provided in the vertical direction (that is, the y-axis direction or the direction perpendicular to the ground) of the scan subject support apparatus 302, or at least two connecting holes or connecting rods are provided in other directions of the scan subject support apparatus 302. The present application is not limited thereto.

In some embodiments, if the docking apparatus 303 is provided with a connecting rod, the scan subject support apparatus 302 is provided with a connecting hole, and the number of connecting rods of the docking apparatus 303 is not greater than the number of connecting holes of the scan subject support apparatus 302. If the docking apparatus 303 is provided with a connecting hole, the scan subject support apparatus 302 is provided with a connecting rod, and the number of connecting holes of the docking apparatus 303 is not less than the number of connecting rods of the scan subject support apparatus 302.

In some embodiments, the connecting hole or the connecting rod of the scan subject support apparatus 302 has a length that exceeds a predetermined threshold, so as to fix the scan subject support apparatus 302 in the transverse direction (that is, the z-axis direction) and the vertical direction (that is, the y-axis direction) of the scan subject support apparatus 302.

For example, the threshold may be correlated with the sum of the weight of the scan subject support apparatus 302 and an approximate weight of the scan subject, and/or be correlated with the position of the center of gravity of the entire apparatus when the movable support portion 3024 reaches a farthest side after moving out of the scan subject support apparatus 302. For example, if the weight of the entire apparatus is large and the center of gravity is greatly shifted after moving out, then the connecting rod may be long, to be capable of load balancing.

In another example, the connecting rod may be made of a material having a high rigidity, for example, a metal column, a metal rod, or a metal pipe (a steel pipe or an iron rod), or may be a high-strength plastic column, plastic rod, plastic pipe, etc. The use of the connecting rod and the connecting hole in cooperation generates a large moment of force, which ensures load balancing even when the movable support portion 3024 reaches the farthest side after moving out of the scan subject support apparatus 302.

Therefore, after docking between the scan subject support apparatus 302 and the docking apparatus 303 is completed, when the movable support portion 3024 at least partially exceeds the range of the support platform 3021 in the up-down direction, the stability of the scan subject support apparatus 302 can be maintained based on the action of the moment of force, and the scan subject support apparatus 302 can be prevented from overturning or moving.

The following further describes the medical imaging system 300 from the side of the docking apparatus 303.

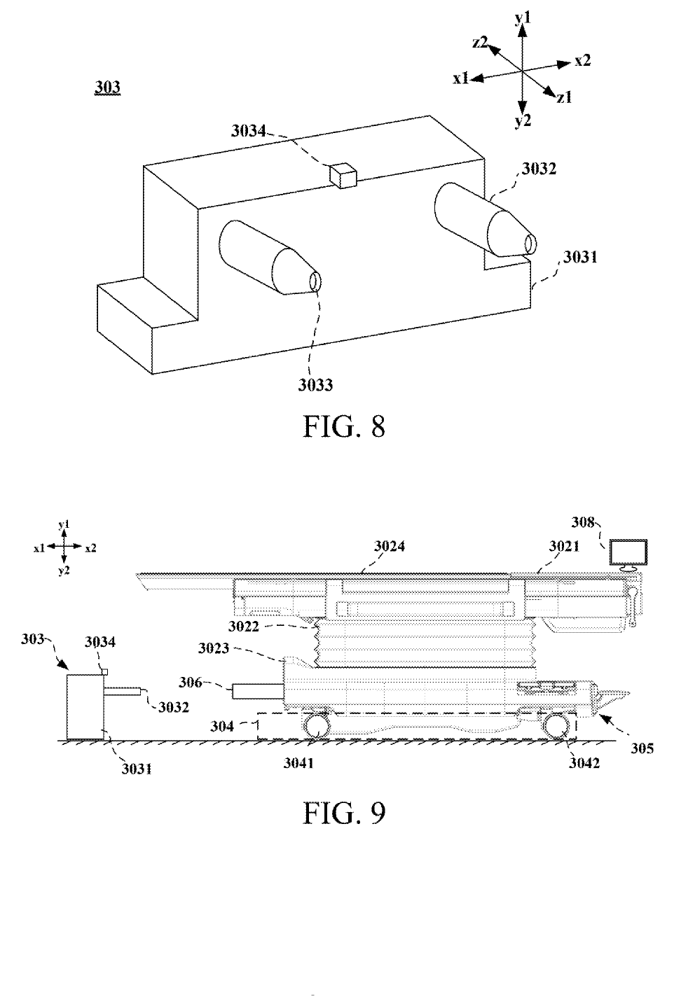

FIG. 8 is a schematic diagram of a docking apparatus according to an embodiment of the present application. As shown in FIG. 8, the docking apparatus 303 includes: a main body portion 3031, fixed to the ground or the scanning apparatus 301, and having a connecting rod or a connecting hole provided in the longitudinal direction of the scan subject support apparatus 302. That is, the main body portion 3031 has a connecting rod or a connecting hole provided in the x-axis direction of the scan subject support apparatus 302. The connecting rod or the connecting hole of the docking apparatus 303 can be used in cooperation with the connecting hole or the connecting rod of the scan subject support apparatus 302. FIG. 8 only schematically shows the connecting rod 3032 of the main body portion 3031. The present application is not limited thereto.

In some embodiments, at least two connecting rods or connecting holes are provided in a transverse direction (that is, the z-axis direction) of the main body portion 3031, or at least two connecting rods or connecting holes are provided in a vertical direction (that is, the y-axis direction or the direction perpendicular to the ground) of the main body portion 3031, or at least two connecting rods or connecting holes are provided in other directions of the main body portion 3031. The present application is not limited thereto.

In some embodiments, if the main body portion 3031 is provided with a connecting rod, the scan subject support apparatus 302 is provided with a connecting hole, and the number of connecting rods of the main body portion 3031 is not greater than the number of connecting holes of the scan subject support apparatus 302. If the main body portion 3031 is provided with a connecting hole, the scan subject support apparatus 302 is provided with a connecting rod, and the number of connecting holes of the main body portion 3031 is not less than the number of connecting rods of the scan subject support apparatus 302.

Therefore, when the scan subject support apparatus 302 moves toward the scanning apparatus 301, the scan subject support apparatus 302 can be smoothly docked with the docking apparatus 303, so as to maintain the stability of the scan subject support apparatus 302 and prevent the scan subject support apparatus 302 from overturning.

In some embodiments, the connecting hole or the connecting rod of the main body portion 3031 has a length that exceeds a predetermined threshold, so as to fix the scan subject support apparatus 302 in the transverse direction (that is, the z-axis direction) and the vertical direction (that is, the y-axis direction or the direction perpendicular to the ground) of the main body portion 3031.

Therefore, after docking between the scan subject support apparatus 302 and the docking apparatus 303 is completed, when the movable support portion 3024 at least partially exceeds the range of the support platform 3021 in the up-down direction, based on the action of a moment of force brought about by a long connecting rod, the scan subject support apparatus 302 can be prevented from overturning, and the stability of the scan subject support apparatus 302 can be improved.

In some embodiments, as shown in FIG. 8, the connecting rod 3032 of the docking apparatus 303 includes a cylindrical main body portion and a conical head portion. The present application is not limited thereto. For example, the connecting rod 3032 may also be comprised of a stepped main body, and the material and shape of the connecting rod are not limited in the present application.

Therefore, the conical head portion of the connecting rod 3032 can conveniently guide the connecting rod 3032 into the connecting hole 3061 of the scan subject support apparatus 302, and the main body portion of the connecting rod 3032 can provide a sufficient action of a moment of force for the scan subject support apparatus 302 to maintain the stability of the scan subject support apparatus.

In some embodiments, the docking apparatus 303 may be further provided with a camera 3033, for example, a camera 3033 located at a front end of the connecting rod or in the connecting hole, where an image acquired by the camera 3033 is displayed on a display portion 308, so as to guide movement of the scan subject support apparatus 302.

For example, the display portion 308 may display a visual field of view image corresponding to the scan subject support apparatus 302 in real time, and the visual field of view image reflects the movement of the scan subject support apparatus 302.

In some embodiments, the display portion 308 may be disposed on the side of the scan subject support apparatus 302 away from the scanning apparatus 301, that is, the side of the x2 direction of the scan subject support apparatus 302. The present application is not limited thereto. Alternatively, the display portion 308 may also be disposed in the scanning apparatus 301. Therefore, an operator can control the scan subject support apparatus 302 to dock with the docking apparatus 303 based on the image displayed on the display portion 308, thereby further improving the convenience of operation.

In some embodiments, the docking apparatus 303 may further be provided with a docking switch 3034, for example, a docking switch 3034 located on the main body portion 3031. When the scan subject support apparatus 302 and the scanning apparatus 301 are successfully docked, the docking switch 3034 is triggered.

For example, after the connecting rod 3032 of the docking apparatus 303 is inserted into the connecting hole 3061 of the scan subject support apparatus 302, the connecting portion 306 of the scan subject support apparatus 302 contacts the docking switch 3034, and at this time, the docking switch 3034 is triggered. After the docking switch 3034 is triggered, the display portion 308 displays “Docking completed”, “Completed docking”, or other content related to the completion of docking. The present application is not limited thereto.

Therefore, after docking between the scan subject support apparatus 302 and the docking apparatus 303 is completed, an operator may promptly acquire information regarding the completion of docking, so as to perform subsequent operations, thereby improving the efficiency of operation.

An embodiment of the present disclosure further provides a medical imaging system. The system includes a scanning apparatus 301, a scan subject support apparatus 302, and a docking apparatus 303.

FIG. 9 is a schematic diagram of docking of a medical imaging system according to an embodiment of the present application. As shown in FIG. 9, the docking apparatus 303 is provided with a connecting rod 3032, and the scan subject support apparatus 302 is provided with a connecting hole 3061 (not shown in FIG. 9). When the scan subject support apparatus 302 approaches the scanning apparatus 301, that is, when the scan subject support apparatus moves in the x1 direction, the camera 3033 (not shown in FIG. 9) located at the front end of the connecting rod 3032 acquires an image and displays the image on the display portion 308. Based on the image displayed on the display portion 308, an operator operates the scan subject support apparatus 302 to dock with the docking apparatus 303. When the connecting rod 3032 of the docking apparatus 303 is inserted into the connecting hole 3061 of the scan subject support apparatus 302, the scan subject support apparatus 302 triggers the docking switch 3034, and the display portion 308 displays “Docking completed”. The operator may operate the pressing apparatus 307, so that the pressing apparatus 307 (not shown in FIG. 9) is pressed, and the fixing rod 3051 (not shown in FIG. 9) of the fixing portion 305 is inserted into the fixing hole 3052 (not shown in FIG. 9) located on the ground.

Therefore, the medical imaging system 300 may fix the scan subject support apparatus 302 based on the action of the moment of force provided by the connecting rod 3032 to restrict the movement of the scan subject support apparatus in the y-axis direction/z-axis direction, and may restrict the movement of the scan subject support apparatus in the x-axis direction based on the fixing rod 3051. Therefore, the stability of the scan subject support apparatus 302 can be improved, preventing problems of failure to complete scanning or even collision with the scanning apparatus caused by tilting of the scan subject support platform.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

What is claimed is:

1. A scan subject support apparatus comprising:
a support platform for bearing a scan subject,
a lifting drive portion for bearing the support platform;
a base for bearing the lifting drive portion:
a moving portion, mounted on the base and capable of moving the entire scan subject support apparatus; and
a connecting portion, provided on the base, the connecting portion including a connecting hole or a connecting rod provided in a longitudinal direction of the scan subject support apparatus; the connecting hole or the connecting rod being capable of being fixedly connected to a docking apparatus fixed to a side of a scanning apparatus.

2. The scan subject support apparatus according to claim 1, wherein,
when the scan subject support apparatus moves toward the scanning apparatus, a connecting rod of the docking apparatus is inserted into the connecting hole of the scan subject support apparatus; and when the scan subject support apparatus is separated from the scanning apparatus, the connecting rod of the docking apparatus leaves the connecting hole of the scan subject support apparatus.

3. The scan subject support apparatus according to claim 1, wherein when the scan subject support apparatus moves toward the scanning apparatus, the connecting rod of the scan subject support apparatus is inserted into a connecting hole of the docking apparatus; and when the scan subject support apparatus is separated from the scanning apparatus, the connecting rod of the scan subject support apparatus leaves the connecting hole of the docking apparatus.

4. The scan subject support apparatus according to claim 1, wherein at least two connecting holes or connecting rods are provided in a transverse direction of the scan subject support apparatus, and the connecting holes or the connecting rods have a length that exceeds a predetermined threshold, so as to fix the scan subject support apparatus in the transverse direction and a vertical direction of the scan subject support apparatus.

5. The scan subject support apparatus according to claim 1, wherein,
the moving portion includes a plurality of casters disposed on a lower side of the base; the plurality of casters include a first caster located on a side of the scan subject support apparatus close to the scanning apparatus during scanning and a second caster located on a side away from the scanning apparatus; and
the connecting portion is disposed at an end of the base close to the scanning apparatus.

6. The scan subject support apparatus according to claim 1, further including a fixing portion, located at an end of the base away from the scanning apparatus; the fixing portion including a fixing rod disposed in a vertical direction and a pressing apparatus capable of moving the fixing rod in the vertical direction.

7. The scan subject support apparatus according to claim 5, wherein the pressing apparatus includes:
a pedal, moving the fixing rod by means of a rotating shaft; and
a guide piece, guiding movement of the fixing rod.

8. The scan subject support apparatus according to claim 5, wherein the ground is provided with a fixing hole;
when the pressing apparatus is pressed, the fixing rod is inserted into the fixing hole to fix the scan subject support apparatus in the longitudinal direction; and when the pressing apparatus is reset to a non-pressed state, the fixing rod is separated from the fixing hole.

9. A docking apparatus, connecting a scan subject support apparatus and a scanning apparatus, the docking apparatus comprising:
a main body portion, fixed to the ground or the scanning apparatus, and having a connecting rod or a connecting hole provided in a longitudinal direction of the scan subject support apparatus, the connecting rod or the connecting hole being capable of being fixedly connected to a connecting hole or a connecting rod of the scan subject support apparatus.

10. The docking apparatus according to claim 8, wherein the connecting rod includes a cylindrical main body portion and a conical head portion; and
at least two connecting holes or connecting rods are provided in a transverse direction of the docking apparatus.

11. The docking apparatus according to claim 8, further including:
a camera, located at a front end of the connecting rod or in the connecting hole,
wherein an image acquired by the camera is displayed on a display portion, so as to guide movement of the scan subject support apparatus.

12. The docking apparatus according to claim 8, further including a docking switch, located on the main body portion; wherein when the scan subject support apparatus and the scanning apparatus are successfully docked, the docking switch is triggered.
